(19) United States
(12) Patent Application Publication
(10) Pub. No.: US 2012/0027640 A1
(43) Pub. Date: Feb. 2, 2012

(54) Disinfecting Method for Climatic Cabinets

(75) Inventors: Stefan Betz, Erlensee (DE); Hubert Heeg, Aschaffenburg (DE); Helmut Loscher, Nidderau (DE)

(73) Assignee: THERMO ELECTRON LED GMBH, Langenselbold (DE)

(21) Appl. No.: 13/013,533

(22) Filed: Jan. 25, 2011

(30) Foreign Application Priority Data
Jan. 26, 2010 (DE) 10 2010 005 748.7

(51) Int. Cl. A61L 2/20; B01L 99/00

(52) U.S. Cl. 422/3; 422/26; 422/500

(57) ABSTRACT

A method for the disinfection of climatic cabinets in particular is provided. The method comprises the following steps: generating a disinfecting hot atmosphere in the space to be disinfected by supplying heat and evaporating water in a heating-up phase; when a temperature of at least 95±5° C. and a relative humidity of at least 80% are reached at a pressure corresponding substantially to the pressure of the ambient atmosphere, maintaining these process conditions in a disinfection phase for a period of time which is sufficient for killing germs situated in the interior space; and subsequently expelling the relative humidity from the space to be disinfected and cooling in a cooling phase, the water vapor generated for disinfection being removed from the space to be disinfected by injecting sterile air in the cooling phase

### CROSS-REFERENCE TO RELATED APPLICATIONS

This application claims priority to foreign Patent Application DE 10 2010 005 748.7, filed on Jan. 26, 2010, the disclosure of which is incorporated herein by reference in its entirety.

### FIELD OF THE INVENTION

The invention relates to a disinfection method, especially for climatic cabinets, and a device for performing the method.

### BACKGROUND OF THE INVENTION

When disinfecting modern climatic cabinets, incubators and breeding apparatus with carousel-like object carrier elements and transport systems which are controlled by sensors as well as by computers and robots, it is necessary to clean the interior spaces including the installed elements contained therein at regular intervals without subjecting the installed elements to excessive stresses.

In particular modern climatic cabinets are equipped with complex robotics systems which have a very large and fissured surface for storing and removing the containers, e.g. microtiter plates and sample carriers. As a result of the exceptionally good growth conditions for germs and microorganisms during the operation of such devices, the surfaces of the installed elements can also provide a good basis for undesirable microorganisms.

As a result of their complexity, the surfaces are virtually inaccessible for manual cleaning and sterilization so that an automated method is necessary. Conventionally employed disinfection gases such as ethylene oxide and formaldehyde always represent an undesirable risk due to the hazards linked with the same, and gassing with hydrogen peroxide requires a very complex apparatus.

Previously known methods for the disinfection of such systems provide hot-steam sterilization at high temperatures of over 120° C. With respect to modern installed elements with heat-sensitive motors, robots and sensors, however, these methods have proven inadequate in order to keep the installed elements operational on a permanent basis.

Furthermore, a known method for the disinfection of gassing incubators operates at a relatively low temperature of 90° C. and a relative humidity of at least 80%. A condensation phase automatically follows the relatively long disinfection phase of at least 9 hours, during which condensation of the water introduced is achieved by cooling the floor wall. It is further reported in this document that a heating at the door is provided to avoid water condensation on the transparent inner door. Condensation on the sensitive installed elements, however, cannot be prevented because they cannot be heated as a door or a smooth inner wall can. As a result, water will condensate on the sensitive installed elements in the described method and can thus easily lead to an undesirable reduction in the life expectancy of the installed elements.

The disadvantage of all previously known methods for the disinfection or sterilization of generic incubators is thus that they do not offer any possibility of sufficiently protecting the heat and humidity-sensitive installed elements of modern devices in order to avert a reduction in their life expectancy.

### SUMMARY OF THE INVENTION

Embodiments of the present invention advantageously provide a method for the disinfection of climatic cabinets that optimally meets the special requirements concerning heat and humidity-sensitive installed elements such as robotic transport systems, motors and sensors. Moreover, the method should simultaneously be capable of minimizing the length of the disinfection process at relatively low temperatures.

One embodiment of a method for the disinfection of an interior space of a climatic cabinet comprises the following steps:
generating a disinfecting hot atmosphere in the interior space by supplying heat and evaporating water in a heating-up phase;
after a temperature of 95±5° C. and a relative humidity of at least 80% are reached at a pressure corresponding substantially to the pressure of the ambient atmosphere, maintaining these process conditions in a disinfection phase for a period of time which is sufficient for killing germs situated in the interior space; and
subsequently expelling the relative humidity from the interior space and cooling in a cooling phase, the water vapor generated for disinfection being removed from the interior space by injecting sterile air in the cooling phase.

An increased heat sensitization of the germs present can be achieved as a result of the highly humid atmosphere as opposed to a dry atmosphere so that advantageously almost all the germs can be thermally destroyed in a disinfection phase that expediently lasts for several hours. It is useful to schedule the disinfection phase for over night or over the weekend in order to avoid an unnecessary loss of working hours. The method further advantageously avoids noxious disinfection gases or such that require complex apparatus such as ethylene oxide, formaldehyde or hydrogen peroxide.

Moreover, undesirable condensation of water on humidity-sensitive installed elements and on the floor can further be avoided by the injection of sterile air to expel water vapor in the cooling phase. The method thus manages for the first time to avoid reducing the life expectancy of installed elements as a consequence of condensation and the resulting potential corrosion. In order to achieve the desired success, the temperature in the interior space is expediently lowered slowly so that the humidity in the interior space is first reduced by the injection of sterile air to an extent that it is no longer sufficient for significant condensation during the lowering of the temperature beneath the condensation point.

Compared with previous methods with long condensation phases and subsequent post-heating phases during which it is necessary to remove residual condensate, the length of the process can advantageously be further minimized as a result of the expulsion of the water vapor by means of sterile air.

In contrast, the method can be automated very well so that manual intervention by operators is no longer required during the process. Certain measured values in the interior space of the climatic cabinet, especially humidity and temperature, are determined for this purpose. Where appropriate, the pressure within the interior space can also be determined. For these measurement parameters, set values for the various phases of the disinfection process are expediently stored in a memory unit. The determined actual values are compared in an evaluation unit with the corresponding set values. If deviations from the set values are determined, a readjustment expediently occurs by means of a control unit in order to bring the value into the desired range.

Specifically, temperature and humidity in the interior space are measured in step i) and the temperature is increased until the temperature and humidity values indicated in step ii) have been obtained. Preferably, a temperature of precisely 90° is set. Subsequently, e.g. a timer is started which measures the time provided for step ii) (e.g. 5 to 12 hours). Moreover, the temperature and humidity continue to be measured and readjustments continue to be made so that the predetermined conditions are maintained over the time provided for step ii). Naturally, certain corridors can be provided within which a deviation from the predetermined values is permissible. Step ii) is ended after expiry of the predetermined time and step iii) is started. At least the air humidity and, where appropriate, the temperature expediently continue to be measured in the interior space during the cooling phase. Sterile air is injected for a length of time until the humidity has reached a predetermined value. The disinfection process is then ended. Normal operation of the climatic cabinet can then be resumed. The measuring devices required for the measurement, such as thermometer and hygrometer, are usually already provided in conventional climatic cabinets, especially in incubators, for determining these parameters.

The sterile air for the expulsion of the water vapor in the cooling phase can be generated in an advantageous embodiment of the method from the ambient air via sterile filters. A sufficient supply with sterile air can thus be guaranteed in a simple and cost-effective way.

The device to be disinfected is expediently switched off prior to the disinfection process. Any culture material or samples that are present are removed from the space to be disinfected and the water supply for the controlled humidification of the atmosphere during the normal operating phase is removed. Moreover, manual cleaning work can be performed, if necessary.

The method can preferably be used for the disinfection of climatic cabinets, incubators and breeding apparatus comprising electronic installed elements and sensors. A disinfection of the entire used space including all installed elements can be performed carefully specifically in these modern laboratory devices with heat and humidity-sensitive installed elements. Moreover, the length of the process can be considerably reduced, as previously necessary condensation and post-heating phases are made redundant.

Another embodiment comprises a climatic cabinet, specifically one that comprises a carousel-like object storage device and at least one transport apparatus which are controlled and operated by robots and sensors, the climatic cabinet comprising an apparatus for generating and conveying sterile air in order to perform the method. The apparatus for generating sterile air can preferably comprise at least one sterile filter for the sterilization of aspirated ambient air. In this way, the sterile air required for blowing out the water vapor used for disinfection can be generated in a simple and cost-effective way from the ambient air.

In a further embodiment of the climatic cabinet, pressure compensation means can be provided which assimilates the pressure in the interior of the space to be disinfected to the pressure of the ambient atmosphere. A beneficial atmosphere can thus be ensured for the installed elements so that material stresses caused by pressures in the interior different from in the outer atmosphere can be avoided.

### BRIEF DESCRIPTION OF THE DRAWINGS

Embodiments of the invention are explained below in greater detail by reference to the drawings and is not limited by the same. Reference numbers designate the different parts. The drawings schematically show:

FIG. 1 shows a perspective view of a climatic cabinet in accordance with an embodiment of the invention, and

FIG. 2 shows a flow chart for illustrating the course of a disinfection method in accordance with an embodiment of the invention.

### DETAILED DESCRIPTION

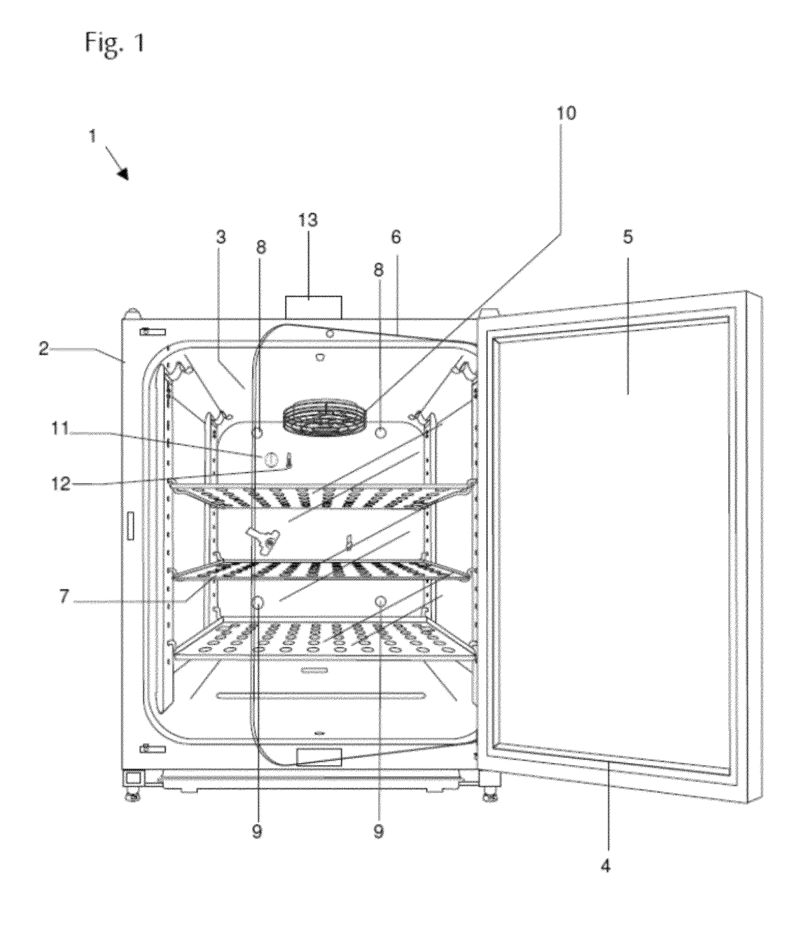

FIG. 1 shows a top view of a climatic cabinet 1 that comprises a cabinet-type housing 2 which encloses an interior space 3. The interior space can be sealed in the direction towards the observer with a door 4. A further door 6 is present between the outside door 4 and the interior space 3 and lies in a depression 5 when both doors are closed. The inner door 6 is made of glass and makes it possible to look into the interior space 3 when the door 4 is open. The inner door 6 prevents an exchange between the atmosphere of the interior space and the ambient environment from occurring too rapidly. The interior space is designed for the storage of samples (not shown here) which can be stored on storage floors 7. Specific temperature, humidity and, where appropriate, gas composition conditions (e.g. a carbon dioxide atmosphere) are generated and maintained in the interior space 3 in order to enable the storage of the samples under optimal conditions or, in the case of an incubator, effecting an incubation of the samples.

Over time, the microbiological samples stored in the interior space lead to a contamination of the interior space. It is therefore necessary to decontaminate the interior space at regular intervals. This occurs by the disinfection of the interior space including all installed elements (thus including the storage floors 7, for example), obviously without samples, by means of hot steam. For this purpose, water (from a water bath disposed in the interior space, for example) is evaporated and heated in a first process section. A fan 10 on the ceiling of the interior space is provided for homogenization of the atmosphere. Humidity and temperature are measured in the interior space with a hygrometer 11 and a thermometer 12. The measured values are sent to a storage and evaluation unit which can be built into the device or arranged externally (very schematically shown here as 13). The measured values are compared there with the stored set values. If the evaluation unit determines that the set values for humidity and temperature predetermined for the heating-up phase have been reached, a control unit (which can be combined with the control and evaluation unit) will give the signal for the start of the second process section.

When a temperature of at least 95±5° C. and a humidity of at least 80% have been reached in the interior space 3, these conditions are maintained for long enough, usually for several hours, until the germs in the interior space have been destroyed. Temperature and relative humidity are measured again and the measured values are compared with the set values in order to perform a temperature adjustment in case of a deviation and to bring the values back into the desired range again. The time is measured as of the beginning of the second phase. The maintaining phase (step ii)) is now performed for the predetermined amount of time. Phase ii) is ended upon expiry of the predetermined time.

There follows the cooling phase during which the climate in the interior space is brought back to normal conditions. Filtered air, preferably sterile air, is injected through air inlet openings 8 into the interior space to expel the hot water vapor from the interior space and the increasingly dehumidified air is removed from the interior space through the outlet openings 9. During this time, the temperature in the interior space gradually cools off. By the measurement of temperature and air humidity in the course of the cooling phase, it can be determined when the predetermined end values have been reached and the disinfection process can be terminated.

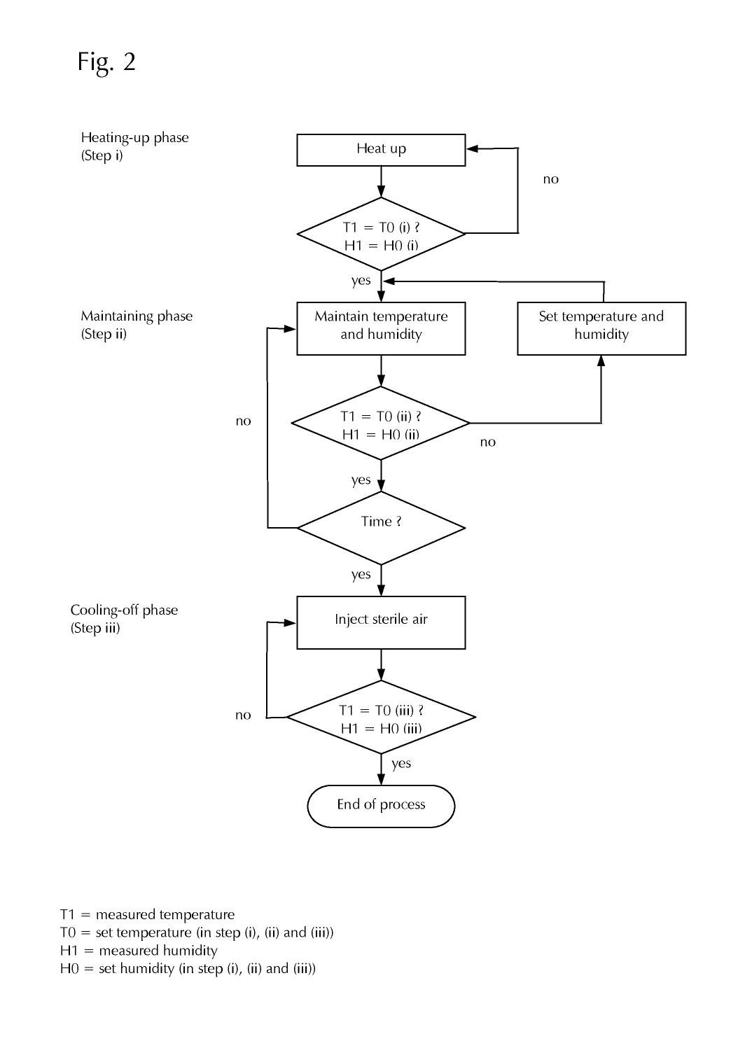

The method steps described above are shown schematically in FIG. 2 in the form of a block diagram.

The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention.

What is claimed is:

1. A method for disinfecting a space, comprising:
i) generating a disinfecting hot atmosphere in the space by supplying heat and evaporating water during a heating-up phase;
ii) when a temperature of at least 95±5° C. and a relative humidity of at least 80% are reached at a pressure corresponding substantially to a pressure of an ambient atmosphere, maintaining these process conditions during a disinfection phase for a period of time which is sufficient for killing germs situated in the space to be disinfected; and
iii) subsequently expelling the relative humidity from the space to be disinfected and cooling during a cooling phase,
wherein the water vapor generated for disinfection is removed from the space to be disinfected by injecting filtered air in the cooling phase.

2. The method according to claim 1, wherein step ii) is performed for a period of several hours.

3. The method according to claim 2, wherein step ii) is performed for a period of 5 to 12 hours.

4. The method according to claim 1, wherein the filtered air for the expulsion of the water vapor in the cooling phase is generated via sterile filters from ambient air.

5. The method according to claim 1, further comprising determining and comparing at least one of the following measured values with a corresponding set value stored in a memory unit during at least one of the steps i), ii) and iii):
temperature in the interior space;
air humidity in the interior space;
pressure in the interior space.

6. The method according to claim 5, further comprising, in cases where a deviation of the measured value from the set value is determined, carrying out a control such that the measured value is assimilated to the set value.

7. The method according to claim 1, wherein the space is an interior space of a climatic cabinet, an incubator or a breeding apparatus, which includes electronic components and sensors.

8. A climatic cabinet, comprising:
an interior space for receiving samples; and
a device for generating and conveying filtered air which is configured to perform a method for disinfecting the interior space, the method comprising:
i) generating a disinfecting hot atmosphere in the space by supplying heat and evaporating water during a heating-up phase;
ii) when a temperature of at least 95±5° C. and a relative humidity of at least 80% are reached at a pressure corresponding substantially to a pressure of an ambient atmosphere, maintaining these process conditions during a disinfection phase for a period of time which is sufficient for killing germs situated in the space; and
iii) subsequently expelling the relative humidity from the space and cooling during a cooling phase,
wherein the water vapor generated for disinfection is removed from the space to be disinfected by injecting filtered air in the cooling phase.

9. A climatic cabinet according to claim 8, further comprising an object storage device and at least one transport apparatus that are controlled and operated by robots and sensors.

10. A climatic cabinet according to claim 9, wherein the object storage device is a carousel-type object storage device.

11. A climatic cabinet according to claim 9, further comprising at least one sterile filter for the sterilization of aspirated ambient air.

12. A climatic cabinet according to claim 8, further comprising pressure compensation means to assimilate a pressure in an interior of the space to be disinfected to a pressure of the ambient atmosphere.

13. An incubator, comprising:
an interior space for receiving samples; and
a device for generating and conveying filtered air which is configured to perform a method for disinfecting the interior space, the method comprising:
i) generating a disinfecting hot atmosphere in the space by supplying heat and evaporating water during a heating-up phase;
ii) when a temperature of at least 95±5° C. and a relative humidity of at least 80% are reached at a pressure corresponding substantially to a pressure of an ambient atmosphere, maintaining these process conditions during a disinfection phase for a period of time which is sufficient for killing germs situated in the space; and
iii) subsequently expelling the relative humidity from the space and cooling during a cooling phase,
wherein the water vapor generated for disinfection is removed from the space to be disinfected by injecting filtered air in the cooling phase.
